(19) United States
(12) Patent Application Publication
(10) Pub. No.: US 2022/0289587 A1
(43) Pub. Date: Sep. 15, 2022

(54) A NOVEL POLYMORPH AND USES THEREOF

(71) Applicant: PROCYPRA THERAPEUTICS, LLC, Mill Valley, CA (US)

(72) Inventors: Daniel J. Carper, Chadds Ford, PA (US); Ashley Kelly Stanley, Darlington, SC (US); Christopher Goss, Florence, SC (US)

(73) Assignee: PROCYPRA THERAPEUTICS, LLC, Mill Valley, CA (US)

(21) Appl. No.: 17/639,649

(22) PCT Filed: Sep. 2, 2020
(86) PCT No.: PCT/US2020/049005

§ 371 Date: Mar. 2, 2022

Related U.S. Application Data
(60) Provisional application No. 62/895,338, filed on Sep. 3, 2019.

(51) Int. Cl. C01G 3/00

(52) U.S. Cl. CPC C01G 3/006; C01P 2002/72

(57) ABSTRACT

In one embodiment, the present application discloses compounds that are selective neuroactive agents for the treatment of diseases of the central nervous system (CNS). In one aspect, the neuroactive agents are compositions comprising Polymorph SP.

### BACKGROUND OF THE APPLICATION

The present invention relates to the use of Cu^{II}-diacetyl-bis (N⁴-methyl-thiosemicarbazone) (also referred to as diacetyl-bis(N⁴-methyl-thiosemicarbazonato)-Cu^{II}, Cu^{II}(atsm), copper ATSM, and CuATSM—the latter term is used herein) as a pharmaceutical agent, in particular for the treatment of conditions in which metal delivery can prevent, alleviate or ameliorate the condition. There are a number of clinical conditions which are caused by or associated with abnormal levels of metals (typically low metal levels). Conditions of this type include cancer and conditions characterized by or associated with oxidative damage, more specifically neurodegenerative conditions or diseases such as Alzheimer's disease (AD), Parkinson's disease (PD), Huntington's disease, hypoxia and prion diseases (PrDs).

Bio-available metals have significant impact on the working of biological systems. It is known that metals play a significant role in enzyme systems and in the signaling mechanisms within biological systems. For example, Zn plays an important role in the ß-amyloid plaques of Alzheimer's disease; the effect of the (Cu, Zn) superoxide dismutase enzyme in mediating reactive oxygen species damage associated with amyotrophic lateral sclerosis; the participation of the heme enzymes NO synthase and guanylyl cyclase in the production and sensing, respectively, of nitric oxide (NO), and the discovery of a “zinc-finger” motif in the breast and ovarian cancer susceptibility gene, BRCA1 for example. In addition, it has been demonstrated that an aberrant protein has a propensity to misfold in the presence of certain concentrations of metal ions.

A number of cardiovascular conditions have been identified that are the result of oxidative stress (OS). Other conditions associated with OS include cancer, cataracts, neurodegenerative disorders such as Alzheimer's disease and heart diseases. There is also evidence that OS plays a prominent role in neuromuscular disorders, including amyotrophic lateral sclerosis (ALS), mitochondrial/metabolic disease, Friedreich's ataxia, Parkinson's disease and Lewy body dementia. Common features of these diseases include the deposition of misfolded protein and substantial cellular damage as a result of OS. Data suggests that OS is the primary cause of physical damage in a wide range of disease states, including amyloidogenic neurological disorders such as Alzheimer's disease (AD), amyotrophic lateral sclerosis (ALS), prion diseases—including Creutzfeldt-Jakob Disease (CJD), transmissible spongioform encephalopathies (TSE), cataracts, mitochondrial disorders, Menkes disease, Parkinson's disease (PD) and Huntington's disease (HD). The effect of OS is not limited to any one part of the human body, with examples of the negative effects of OS being observed for almost all organs. For example, the human brain is an organ that concentrates metal ions and recent evidence suggests that a breakdown in metal homeostasis plays a critical role in a variety of age-related neurodegenerative diseases.

A number of therapeutic agents have been developed as potential therapies for the conditions caused by or associated with OS. However, agents such as vitamin E and vitamin C were found to be ineffective, as they do not cross the blood brain barrier and accordingly, cannot be used effectively for the treatment of neurodegenerative diseases of central origin.

Copper metal ion deficiency has been reported as a condition associated with neurodegenerative diseases such as ALS, PD and Lewy body dementia. One consequence of copper deficiency is that the protective enzymes responsible for detoxifying reactive oxygen species (ROS) are inadequately loaded with copper and therefore do not effectively carry out normal enzyme function. The inadequate loading of such protective enzymes, for example in the brain, leads to a general increase in OS (as is observed in AD) which will be reflected in increased protein oxidation, such as increased protein carbonyls.

Accordingly, there is a need for highly effective agents such as CuATSM for the treatment of disease associated with oxidative damage and particularly central nervous system neurodegenerative disorders such as PD, AD and ALS. In addition, there is a need for novel agents for the treatment of conditions associated with peripheral tissues, gastrointestinal dysfunction such as constipation, and acute respiratory distress syndrome, ALS, atherosclerotic cardiovascular disease and multiple organ dysfunction. There is a further need for a commercially viable process for making such novel agents.

Although it has been shown that CuATSM is effective in the treatment of diseases associated with oxidative damage and particularly central nervous system neurodegenerative disorders such as PD and ALS, CuATSM itself is virtually insoluble in aqueous media. Accordingly, there is a need for the provision of CuATSM in a form that is more soluble in aqueous media and/or more able to be delivered in an orally available formulation.

### SUMMARY OF THE APPLICATION

There is a continuing need for novel and effective agents that are selective neuroactive agents for the treatment of diseases of the central nervous system (CNS). Accordingly, it is a first object of this invention to provide a stable polymorph of CuATSM:gluconic acid. It is a further object of this invention to provide a process for preparation of the stable polymorph, which is safe and commercially viable.

Accordingly, described herein is a stable polymorph of Cu^{II}-diacetyl-bis (N⁴-methyl-thiosemicarbazone) (CuATSM) and gluconic acid, wherein the polymorph has an X-Ray Powder Diffraction (“XRPD”) spectrum comprising peaks at Two-Theta angles of 7.5, 9, and 11 degrees. In some embodiments, the XRPD spectrum comprises additional peaks at 15.5, 27.5, 28.5 and 32 degrees. The polymorph will typically have at least five XPRD spectrum peaks selected from the group consisting of Two-Theta angles of approximately 7.5, 9, 11, 15.5, 27.5, 28.5, and 32 degrees. Alternatively, the polymorph will have at least six XPRD spectrum peaks selected from the group consisting of Two-Theta angles of approximately 7.5, 9, 11, 15.5, 27.5, 28.5, and 32 degrees. The polymorph may have XPRD spectrum peaks at Two-Theta angles of approximately 7.5, 9, 11, 15.5, 27.5, 28.5, and 32 degrees.

Further described herein is a method for the treatment or prophylaxis of a condition in a mammal in which metal delivery prevents, alleviates or ameliorates the condition comprising the step of administering to the mammal a therapeutically effective amount of a composition comprising: a stable polymorph of CuATSM and gluconic acid, wherein the composition has an XRPD spectrum comprising peaks at Two-Theta angles of 7.5, 9, and 11 degrees; and a pharmaceutically acceptable excipient. The composition may comprise a polymorph with at least five XPRD spectrum peaks selected from the group consisting of Two-Theta angles of approximately 7.5, 9, 11, 15.5, 27.5, 28.5, and 32 degrees. Alternatively, the composition may comprise a polymorph with at least six XPRD spectrum peaks selected from the group consisting of Two-Theta angles of approximately 7.5, 9, 11, 15.5, 27.5, 28.5, and 32 degrees. The composition may comprise a polymorph with XPRD spectrum peaks at Two-Theta angles of approximately 7.5, 9, 11, 15.5, 27.5, 28.5, and 32 degrees.

Also described herein is a process for the preparation of a stable polymorph of CuATSM and gluconic acid, the process comprising the steps of (a) mixing ATSMH₂ and copper gluconate (in a first ratio) with a solvent (in a second ratio) to form a slurry, (b) heating the slurry to form a composition comprising Polymorph SP, and (c) isolating the polymorph.

Further described herein is a stable polymorph of CuATSM and gluconic acid made by a process comprising the steps of (a) mixing ATSMH₂ and copper gluconate (in a first ratio) with a solvent (in a second ratio) to form a slurry, (b) heating the slurry to form the composition, and (c) isolating the polymorph.

### BRIEF DESCRIPTION OF THE FIGURES

FIG. 1 is an X-Ray Powder Diffraction (“XRPD”) spectrum for the product made in Example 1. The spectrum for the product is shown at the top of FIG. 1 and compared to the XRPD spectra of CuATSM, copper D-gluconate, D-gluconic acid lactone, and D-gluconic acid respectively, below.

FIG. 2 is an XRPD spectrum for the product made in Example 2. The spectrum for the product is shown at the top of FIG. 2 and compared to the XRPD spectra of CuATSM, copper D-gluconate, and D-gluconic acid lactone respectively, below.

FIG. 3 is an XRPD spectrum for the product made in Example 3. The spectrum for the product is shown at the top of FIG. 3 and compared to the XRPD spectra of CuATSM, copper D-gluconate, and D-gluconic acid lactone respectively, below.

FIG. 4 is an XRPD spectrum for the product made in Example 4. The spectrum for the product is shown at the top of FIG. 4 and compared to the XRPD spectra copper D-gluconate and D-gluconic acid lactone respectively, below.

### DETAILED DESCRIPTION OF THE APPLICATION

The present application discloses stable polymorphs of a composition comprising CuATSM and gluconic acid. CuATSM (also known as Cu^{II}-diacetyl-bis (N⁴-methyl-thiosemicarbazone), diacetyl-bis(N⁴-methyl-thiosemicarbazonato)-Cu^{II}, Cu^{II}(atsm), or copper ATSM) has the following structure:

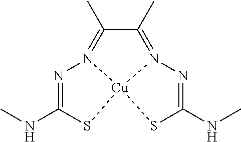

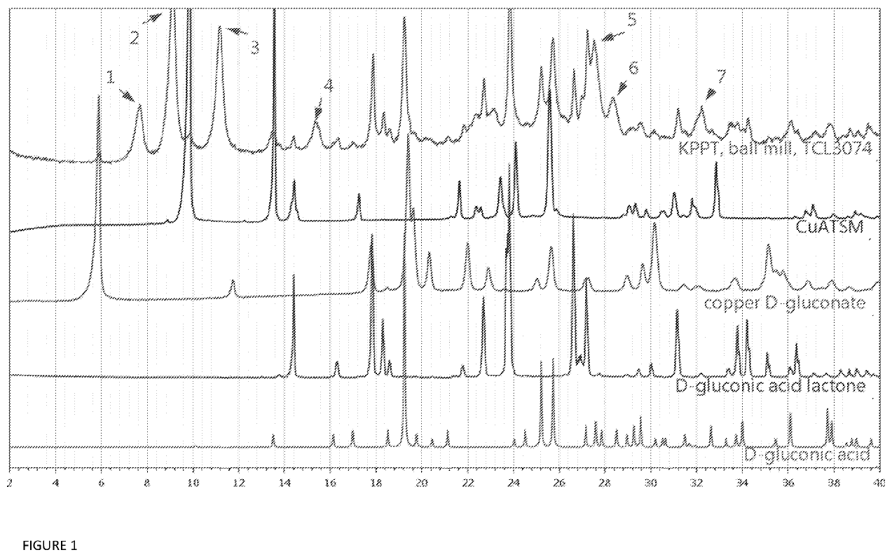

The stable polymorph of CuATSM and gluconic acid is also referred to herein as Polymorph SP. The stable polymorph has a unique XRPD spectrum as disclosed herein. In some embodiments, the stable polymorph has an XRPD spectrum comprising peaks at Two-Theta angles of 7.5, 9, and 11 degrees. In some embodiments, in addition to those peaks, the stable polymorph has an XRPD spectrum that further comprises peaks at Two-Theta angles of 15.5, 27.5, 28.5 and 32 degrees. The “Two-Theta angle” values given herein are meant to be best approximations of the peak values shown at the top of FIGS. 1, 3 and 4 herein.

The present application discloses the use of Polymorph SP to deliver copper metal to biological sites, tissues or cells wherein copper is depleted in a patient. A number of important copper-mediated biological processes, such as copper-mediated enzymatic processes, occur in the cells rather than in the extra-cellular matrix. Copper is delivered in the form of Polymorph SP to ensure that copper acts in the cell rather than in the extra-cellular environment. In addition, Polymorph SP delivers copper to the cell such that upon administration to a patient copper is not released in the extra-cellular environment.

The properties of CuATSM in Polymorph SP are typically retained on dissolution of the polymorph, such that the inherent properties of CuATSM, including but not limited to cellular uptake, bioavailability, ability to cross the blood-brain-barrier, redox potential, or therapeutic efficacy, are maintained.

### Definitions

Unless specifically noted otherwise herein, the definitions of the terms used are standard definitions used in the art of organic synthesis and pharmaceutical sciences. Exemplary embodiments, aspects and variations are illustrated in the figures and drawings, and it is intended that the embodiments, aspects and variations, and the figures and drawings disclosed herein are to be considered illustrative and not limiting.

The term “neurodegenerative disorder” refers to an abnormality in which neuronal integrity is threatened. Neuronal integrity can be threatened when neuronal cells display decreased survival or when the neurons can no longer propagate a signal. Neurological conditions that can be treated with the compositions comprising Polymorph SP of the present application include the conditions as recited herein.

The term “neurological condition” refers to conditions in which various cell types of the nervous system are degenerated and/or have been damaged as a result of neurodegenerative disorders or injuries or exposures. In particular, the compositions comprising Polymorph SP of the present application may be used for the treatment of resulting conditions, in which damage to cells of the nervous system has occurred due to surgical interventions, infections, exposure to toxic agents, tumours, nutritional deficits or metabolic disorders. In addition, the compositions comprising Polymorph SP may be used for the treatment of the sequel of neurodegenerative disorders, such as Alzheimer's disease, Parkinson's disease, multiple sclerosis, amyotrophic lateral sclerosis, epilepsy, drug abuse or drug addiction (alcohol, cocaine, heroin, amphetamine or the like), spinal cord disorders, dystrophy or degeneration of the neural retina (retinopathies) and peripheral neuropathies, such as diabetic neuropathy and/or the peripheral neuropathies induced by toxins.

The term “patient” as used herein refers to any animal having a disease or condition which requires treatment or prophylaxis with a biologically-active agent. The patient may be a mammal, such as a human, or may be a non-human primate or non-primates such as used in animal model testing. While the compounds are suitable for use in medical treatment of humans, it is also applicable to veterinary treatment.

The phrase “pharmaceutically acceptable” means that the compound, substance or composition is compatible chemically and/or toxicologically with the other ingredients comprising a formulation, and/or with the patient being treated.

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of a disease state.

Generally, the terms “treatment” and “prophylaxis” mean affecting a subject, tissue or cell to obtain a desired pharmacological and/or physiological effect and include: (a) preventing a condition from occurring in a subject that may be predisposed to the condition, but has not yet been diagnosed as having it; (b) inhibiting the condition, i.e., arresting its development; or (c) relieving or ameliorating the effects of the condition, i.e., cause regression of the effects of the condition.

### Methods of Making CuATSM

Methods for the preparation of metal complexes such as CuATSM and their methods for the treatment of various neurodegenerative diseases and disorders, are disclosed in PCT/AU2007/001792, published as WO2008/061306, which is incorporated herein in its entirety. Representative examples are provided herein.

CuATSM may also be prepared using the reaction routes and synthesis schemes as described below, employing the techniques available in the art for each of the individual step/reactions and using starting materials that are readily available. Suitable protecting groups can be found in T. W. Greene's Protective Groups in Organic Synthesis, John Wiley & Sons, 1981.

Preparation of a complex such as CuATSM is shown in this scheme:

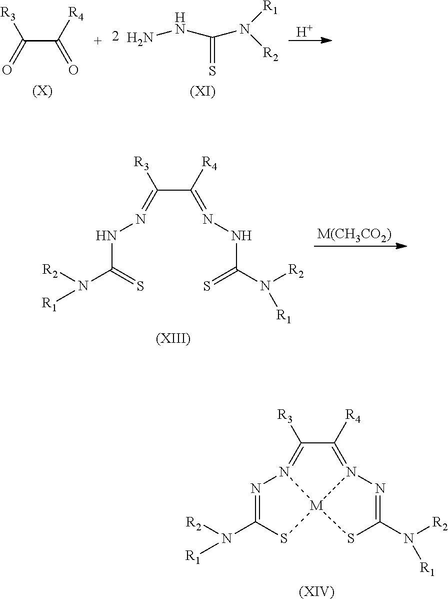

Condensation of dione (X) with two equivalents of a suitably functionalized thio semicarbazide (XI) under acidic conditions leads to the formation of the bis (thiosemicarbazone) (XIII). The bis(thiosemicarbazone) can then be reacted with a suitable metal salt such as the metal acetate to produce the desired metal complex (XIV) and acetic acid.

### Methods of Making a Novel Polymorph of CuATSM and Gluconic Acid

The methods for making Polymorph SP begin with ATSMH₂ (also called ATSM-free ligand):

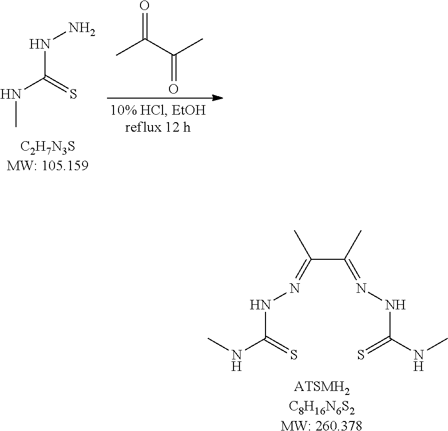

(also known as (2E,2′E)-2,2′-(butane-2,3-diylidene)bis(N-methylhydrazine-1-carbothioamide)) and copper gluconate:

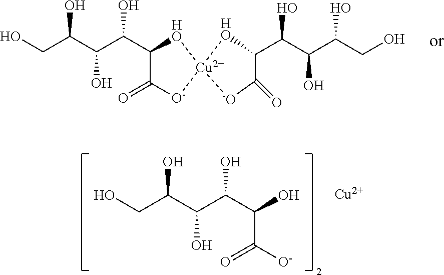

However, when mixed together, the resultant composition is a mixture of CuATSM and gluconic acid. In aqueous solution, gluconic acid will be in equilibrium with its lactone form, gluconolactone. Gluconic acid exists in a (D)- and (L)-form, and mixtures (including racemic mixtures) thereof. In some embodiments, the resultant composition of Polymorph SP is a mixture of CuATSM and D-gluconic acid.

A process for the preparation of Polymorph SP may comprise the steps of: mixing ATSMH₂ and copper gluconate in a solvent to form a slurry; heating the slurry for a programmed time course to form Polymorph SP; and isolating the polymorph.

The above process may also be used to make Polymorph SP starting with CuATSM and D-gluconic acid (instead of ATSMH₂ and copper gluconate). Polymorph SP can also be made using a ball-mill method, as shown in Example 1 below.

In the first step, ATSMH₂ and copper gluconate are mixed together in a solvent to form a slurry. The ATSMH₂ and copper gluconate are added in a molar ratio between 2:1 and 1:2. In some embodiments they are added in a molar ratio between 1.05:1 and 1:1.05. In still other embodiments, they are added in a molar ratio of about 1:1.

The solvent is selected from the group consisting of C₆-C₁₀ alkanes or cycloalkanes, C₁-C₆ alkyl acetates, and mixtures thereof. In some embodiments, the alkane or cycloalkane is hexane, heptane or cycloheptane. In some embodiments, the alkyl acetate is methyl acetate, ethyl acetate, isopropyl acetate, or t-butyl acetate. In some embodiments, the solvent is heptane, isopropyl acetate, or a heptane/isopropyl acetate mixture.

The slurry is then heated slowly from 40° C. to 80° C. In some embodiments, the heating process is done progressively in 20° C. increments over 15-25 days until the reaction is complete by HPLC analysis (≤2% ATSMH₂).

The Polymorph SP is then isolated. In some embodiments, isolation is performed by filtration or centrifugation. In some embodiments, isolation is performed by vacuum filtration, followed by washing with heptane.

Methods of Treatment, Ameliorating and/or Prophylaxis

Compositions comprising Polymorph SP are effective as copper metal delivery agents, particularly agents for the delivery of copper to cells. Compositions comprising Polymorph SP may be used in the treatment or prophylaxis of a number of conditions in which metal delivery can prevent, alleviate or ameliorate the condition. There are a number of conditions of this type. Examples of conditions of this type are conditions associated with or caused by oxidative stress. It is known that many of the protective biological anti-oxidant mechanisms involve copper-catalysed enzymes and thus copper delivery can serve to stimulate or re-start the activity of the biological anti-oxidant mechanisms leading to an overall anti-oxidant effect being achieved. In one embodiment the condition associated with or caused by oxidative stress is selected from the group consisting of cardiovascular conditions, cancers, cataracts, neurological disorders such as Alzheimer's disease, prion diseases—including Creutzfeldt-Jakob Disease (CJD), and heart diseases, amyloidogenic amyotrophic lateral sclerosis (ALS), prion transmissible spongioform encephalopathies (TSE), cataracts, mitochondrial disorders, Menkes disease, Parkinson's disease and Huntington's disease.

In another embodiment the disorder is a neuromuscular disorder selected from the group consisting of amyotrophic lateral sclerosis (ALS), mitochondrial/metabolic disease and Friedreich's ataxia. In one embodiment, the condition is a neurological condition or a neurodegenerative disorder.

Additionally, compositions comprising Polymorph SP may also be used to potentiate the effects of other treatments, for example to potentiate the neuroprotective effects of brain derived nerve growth factor. Compositions comprising Polymorph SP may also be used to treat Anemia, Neutropenia, Copper deficiency Myelopathy, Copper deficiency Syndrome and Hyperzincemia. In addition, the method of treatment is also directed to conditions which induce oxidative damage of the central nervous system, including acute and chronic neurological disorders such as, cerebral ischemia, stroke (ischemic and hemorragic), subarachnoid hemorrhage/cerebral vasospasm, cerebral tumour, AD, CJD and its new variant associated with “mad cow” disease, HD, PD, Friedrich's ataxia, cataract, dementia with Lewy body formation, multiple system atrophy, Hallerboden-Spatz disease, diffuse Lewy body disease, amyotrophic lateral sclerosis, motor neuron disease, multiple sclerosis, fatal familial insomnia, Gertsmann Straussler Sheinker disease and hereditary cerebral hemorrhage with amyloidoisis-Dutch type.

The method of treatment is also directed to the treatment of neurodegenerative amyloidosis. The neurodegenerative amyloidosis may be any condition in which neurological damage results from the deposition of amyloid. The amyloid may be formed from a variety of protein or polypeptide precursors, including but not limited to Aβ, synuclein, Huntington or prion protein. In one embodiment, the condition is selected from the group consisting of sporadic or familial AD, ALS, motor neuron disease, cataract, PD, Creutzfeldt-Jacob disease and its new variant associated with “mad cow” disease, HD, dementia with Lewy body formation, multiple system atrophy, Hallerboden-Spatz disease, and diffuse Lewy body disease.

In another embodiment the neurodegenerative amyloidosis is an Aβ-related condition, such as AD or dementia associated with Down syndrome or one of several forms of autosomal dominant forms of familial AD (reviewed in St George-Hyslop, 2000). Most preferably the Aβ-related condition is AD. In another embodiment, prior to treatment the patient may have moderately or severely impaired cognitive function, as assessed by the AD Assessment Scale (ADAS)-cog test, for example an ADAS-cog value of 25 or greater. In addition to slowing or arresting the cognitive decline of a subject, compositions comprising Polymorph SP and the methods of the invention may also be suitable for use in the treatment or prevention of neurodegenerative conditions, or may be suitable for use in alleviating the symptoms of neurodegenerative conditions. If administered to a patient who has been identified as having an increased risk of a predisposition to neurodegenerative conditions, or to a subject exhibiting pre-clinical manifestations of cognitive decline, such as Mild Cognitive Impairment or minimal progressive cognitive impairment, these compositions comprising Polymorph SP and their methods of use may be able to prevent or delay the onset of clinical symptoms, in addition to the effect of slowing or reducing the rate of cognitive decline.

The compositions comprising Polymorph SP of the present application may also be useful to treat cancer. The term “cancer” describes any array of different diseases linked by cumulative multiple genetic mutations, which result in the activation of oncogenes and/or the inactivation of tumor suppressor genes and/or linked by uncontrolled cellular proliferation. The cause and source of these mutations differs between different cancers of human body organs.

In one embodiment, the present application is directed to a method of treating brain cancer, which includes a brain tumor. A brain cancer or tumor may be a glioma or non-glioma brain tumor. As used herein, the term “cancer” and “tumor” may be used interchangeably herein. “Cancer” may include any one of the following states: glioma, adenoma, blastoma, carcinoma, sarcoma and inclusive of any one of Medulloblastoma, Ependymoma, Astrocytoma, Optical nerve glioma, Brain stem glioma, Oligodendroglioma, Gangliogliomas, Craniopharyngioma or Pineal Region Tumors. Reference to a “glioma” includes GMB, astrocytoma and anaplastic astrocytoma or related brain cancers.

Compositions comprising Polymorph SP of the present application may also be used to treat tau-related disorders. Tau protein is an important protein as it is the protein expressed in the central nervous system and plays a critical role in the neuronal architecture by stabilizing intracellular microtubule network. Thus, any impairment of the physiological role of the tau protein either by truncation, hyper-phosphorylation or by disturbing the balance between the six naturally occurring tau isoforms is detrimental to the subject and leads to the formation of neurofibrillary tangles (NFT), dystrophic neurites and neuropil threads. The major protein subunit of these structures is microtubule associated protein tau. The amount of NFT found in autopsies of AD patients correlates with clinical symptoms including intellectual decline. Accordingly tau protein plays a critical role in AD pathology.

It is believed that the activity of compositions comprising Polymorph SP of the present application that reduce the levels of tau phosphorylation is as a result of their ability to deliver metal to cells and hence their anti-oxidant activity. The complexes act as anti-oxidants may mean that they provide protection from OS which is desirable as OS can lead to hyper-phosphorylation of tau and cell dysfunction. As a consequence the ability of these complexes to deliver biologically important metals to cells allows them to function as anti-oxidants (especially where the oxidative stress is caused by metal deficiency) which in turn means the metal complexes may have the ability to prevent (or treat) tau-opathies. There are a number of disorders or conditions that are recognized as being tau disorders or more colloquially Tauopathies. Disorders of this type include Richardson's syndrome, Progressive Supranuclear Palsy, Argyrophilic grain disease, corticobasal degeneration, Pick's disease, frontotemporal dementia linked with parkinsonism linked to chromosome 17 (FTDP-17), post-encephalitic parkinsonism (PEP), dementia pugilistica, Down syndrome, Alzheimer's disease, Familial British dementia, Familial Danish dementia, Parkinson's disease, Parkinson's Disease complex of Guam (PDC), myotonic dystrophy, Hallevorden-Spatz disease and Niemann-Pick type C.

Compositions comprising Polymorph SP may also be used in the treatment of an Abeta related disorder. A number of Abeta disorders are known including disorders selected from the group consisting of Parkinson's disease, Alzheimer's disease, Multiple sclerosis, Neuropathies, Huntington's disease, Prion disease, motor neuron disease, Amyotrophic lateral sclerosis (ALS), Menkes disease and amyloidoses.

As the compositions comprising Polymorph SP have also been shown to be able to deliver copper to cells they have the ability to influence matrix metallo-proteinases (MMP's). Matrix metalloproteinases (MMPs) are a family of zinc- and calcium-dependent secreted or membrane anchored endopeptidases which play a number of important biological functions. MMPs are involved in many physiological processes but also take part in the pathophysiological mechanisms responsible for a wide range of diseases. Pathological expression and activation of MMPs are associated with cancer, atherosclerosis, stroke, arthritis, periodontal disease, multiple sclerosis and liver fibrosis.

In addition to slowing or arresting the cognitive decline of a subject, compositions comprising Polymorph SP and the methods of the invention may also be suitable for use in the treatment, prevention or alleviation of gastrointestinal (GI) disease or disorder, such as constipation. If administered to a patient who has been identified as having an increased risk of a predisposition to neurodegenerative conditions and GI disease or disorder, or to a subject exhibiting pre-clinical manifestations of cognitive decline and associated GI disease or disorder, these metal complexes and their methods of use may be able to prevent or delay the onset of clinical symptoms, in addition to the effect of slowing or reducing the rate of cognitive decline, along with the treatment prevention or alleviation of the GI disease or disorder. While certain proposed mechanism of action are noted herein, the inventors do not intend to bound by any proposed or suggested mechanism of action in the present invention.

In one aspect, compositions comprising Polymorph SP may be administered via oral or non-oral methods, to a mammal without requiring the formulation with excipients, solubilizers and the like that are not acceptable for human use.

### Administration of Compositions Comprising Polymorph SP

Administration of the compositions comprising Polymorph SP to humans can be performed by any of the accepted modes of administration well known in the art. For example they may be administered by enteral administration such as oral or rectal, or by parenteral administration such as subcutaneous, intramuscular, intravenous and intradermal routes. Injection can be bolus or via constant or intermittent infusion. The composition comprising Polymorph SP is typically included in a pharmaceutically acceptable carrier or diluent and in an amount sufficient to deliver to the subject a therapeutically effective dose.

The compositions comprising Polymorph SP may be administered in any form or mode which makes the complex bio-available. One skilled in the art of preparing formulations can readily select the proper form and mode of administration depending upon the particular characteristics of the complex selected, the condition to be treated, the stage of the condition to be treated and other relevant circumstances. See Remingtons Pharmaceutical Sciences, 19^{th} edition, Mack Publishing Co. (1995). In one aspect, compositions comprising Polymorph SP can be administered alone or in the form of a pharmaceutical composition in combination with a pharmaceutically acceptable carrier, diluent or excipient.

Pharmaceutical compositions comprising Polymorph SP for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. These compositions comprising Polymorph SP may also contain adjuvants such as preservative, wetting agents, emulsifying agents and dispersing agents.

Solid dosage forms for oral administration include capsules, tablets, pills, powders, and granules. In such solid dosage forms, the active complex is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose and acacia, c) humectants such as glycerol, d) disintegrating agents such as agar-agar, calcium carbonate, potato or tapioca starch, alginic acid, certain silicates and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

### EXAMPLES

### X-Ray Powder Diffraction (XRPD) Methodology

A Rigaku Smart-Lab X-ray diffraction system was configured for reflection Bragg-Brentano geometry using a line source X-ray beam. The x-ray source was a Cu Long Fine Focus tube that was operated at 40 kV and 44 mA. That source provides an incident beam spectrum at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1° 2θ or less. The axial divergence of the X-ray beam is controlled by 5.0-degree Soller slits in both the incident and diffracted beam paths.

Samples were placed in low-background, silicon holders using light manual pressure to keep the sample surfaces flat and level with the reference surface of the holder. Each sample was analyzed from 2 to 40° 2θ using a continuous scan of 6° 2θ per minute with an effective step size of 0.02° 2θ.

### Example 1

### Ball Mill Bulk Synthesis of Polymorph SP

In a PK blender were placed pre-weighed solids of ATSMH₂ (1000.0 g) and copper(II) D-gluconate (1750.0 g). The solids were blended for 10 minutes. The resulting mixed powder was transferred to a ball mill with a 10-liter porcelain jar (Shimpo) containing one-inch media balls. The ball mill was activated and mixing begun, inspecting frequently until a dark copper-brown color was obtained. This occurred after 17.75 days of mixing. This process yielded 2368.8 g of product (86% yield, polymorph SP). The XRPD spectrum for this product is shown at the top of FIG. 1 and compared to the XRPD spectra of CuATSM, copper D-gluconate, D-gluconic acid lactone, and D-gluconic acid. The XRPD spectra for Polymorph SP shows unique peaks at approximately 7.5, 9, 11, 15.5, 27.5, 28.5, and 32 that do not appear in the XRPD spectra for CuATSM free base, copper D-gluconate, D-gluconic acid lactone, and D-gluconic acid.

### Example 2

### IPA Slurry Synthesis

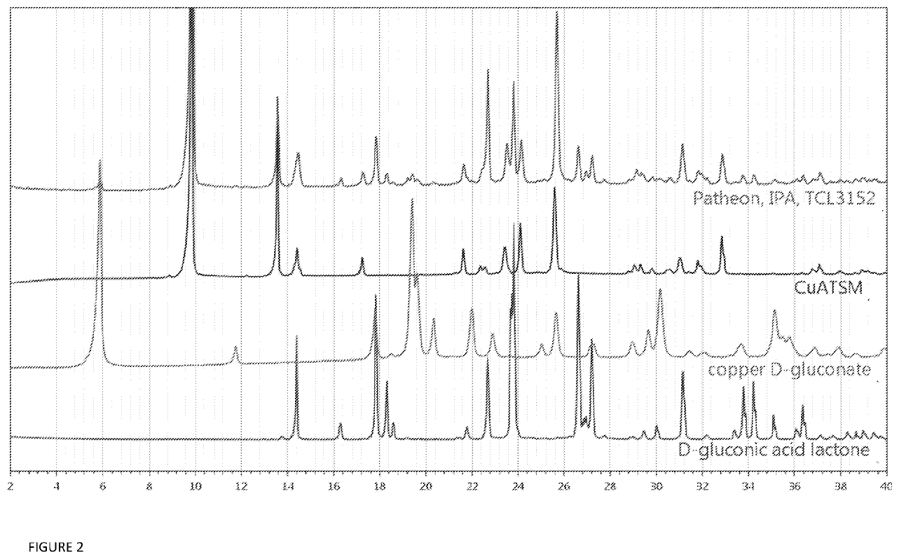

ATSMH₂ (1 eq) and Cu(II) gluconate (1 eq) were suspended in isopropyl alcohol (20 v) and agitated at 40° C. for 7 d, warmed at 60° C. for 8 d, and then 80° C. for 2 d until the reaction was complete. The brown slurry was isolated via filtration, washing with heptane (2×2 vol) and drying in a vacuum oven to afford CuATSM gluconate (100% yield, NOT polymorph SP) as a brown solid. The XRPD spectrum for this product is shown at the top of FIG. 2 and compared to the XRPD spectra of CuATSM, copper D-gluconate, and D-gluconic acid lactone. As shown by the XRPDs, this method does not yield the CuATSM:gluconic polymorph with unique peaks at approximately 7.5, 9, 11, 15.5, 27.5, 28.5, and 32.

### Example 3

### Heptane Slurry Synthesis of Polymorph SP

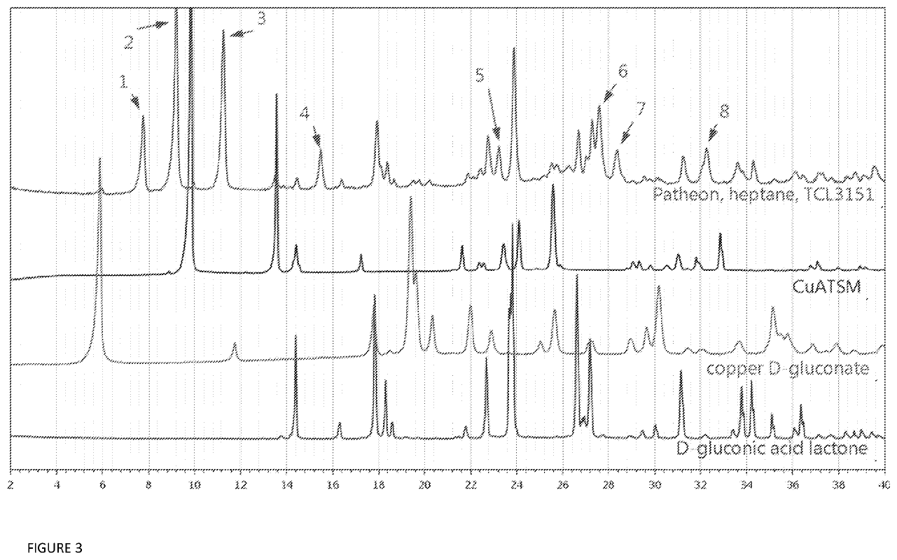

ATSMH₂ (1 eq) and Cu(II) gluconate (1 eq) were suspended in heptane (20 v) and agitated at 40° C. for 7 d, warmed at 60° C. for 8 d, and then 80° C. for 2 d until the reaction was complete. The brown slurry was isolated via filtration, washing with heptane (2×2 vol) and drying in a vacuum oven to afford CuATSM gluconate (100% yield, polymorph SP) as a brown solid. The XRPD spectrum for this product is shown at the top of FIG. 3 and compared to the XRPD spectra of CuATSM, copper D-gluconate, and D-gluconic acid lactone. The XRPD spectra for Polymorph SP shows unique peaks at approximately 7.5, 9, 11, 15.5, 27.5, 28.5, and 32 that do not appear in the XRPD spectra for CuATSM free base, copper D-gluconate, D-gluconic acid lactone, and D-gluconic acid.

### Example 4

### Isopropyl Acetate Slurry Synthesis of Polymorph SP

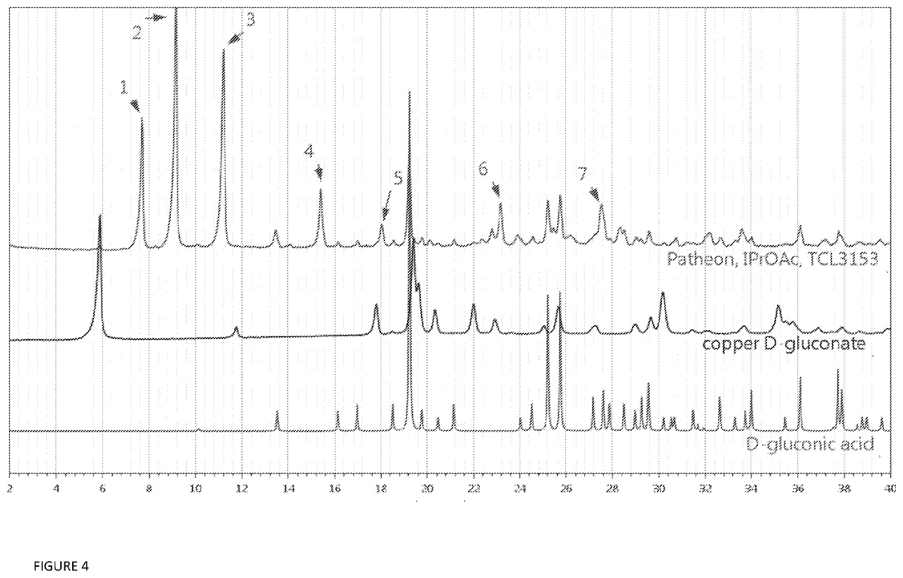

ATSMH₂ (1 eq) and Cu(II) gluconate (1 eq) were suspended in isopropyl acetate (20 v) and agitated at rt for 8 d until the reaction was complete. The brown slurry was concentrated in vacuo (10 v) isolated via filtration, washing with isopropyl acetate (2×2 vol) and drying in a vacuum oven to afford CuATSM gluconate (94% yield, polymorph SP) as a brown solid. The XRPD spectrum for this product is shown at the top of FIG. 4 and compared to the XRPD spectra of copper D-gluconate and D-gluconic acid. The XRPD spectra for Polymorph SP shows unique peaks at approximately 7.5, 9, 11, 15.5, 27.5, 28.5, and 32 that do not appear in the XRPD spectra for CuATSM free base, copper D-gluconate, D-gluconic acid lactone, and D-gluconic acid.

### Example 5

### Heptane/Isopropyl Acetate Slurry Synthesis of Polymorph SP

ATSMH₂ (1 eq) and Cu(II) gluconate (1 eq) were suspended in heptane (15 v) and isopropyl acetate (15 v) and agitated at 40° C. for 7 d, warmed at 60° C. for 7 d, and then 80° C. for 4 d until the reaction was complete. The brown slurry was isolated via filtration, washing with heptane (2×2 vol) and drying in a vacuum oven to afford CuATSM gluconate (90% yield, polymorph SP) as a brown solid. The XRPD spectrum for this product comprises unique Two-Theta peaks at angles of 7.5, 9, 11, 15.5, 27.5, 28.5 and 32 degrees that do not appear in the XRPD spectra for CuATSM free base, copper D-gluconate, D-gluconic acid lactone, and D-gluconic acid.

The XRPD spectra discussed above demonstrate that the products made in Examples 1, 3, 4 and 5 have unique spectra with Two-Theta peaks at angles of 7.5, 9, 11, 15.5, 27.5, 28.5 and 32 degrees, while the spectra for product made in Example 2, the precursor materials CuATSM and copper D-gluconate (and D-gluconic acid and D-gluconic acid lactone, for that matter) do not contain these peaks.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope. The entire disclosures of all documents cited throughout this application are incorporated herein by reference.

What is claimed is:

1. A stable polymorph of Cu^{II}-diacetyl-bis (N⁴-methyl-thiosemicarbazone) (CuATSM) and gluconic acid, wherein the composition has at least five XRPD spectrum peaks selected from the group consisting of Two-Theta angles of approximately 7.5, 9, 11, 15.5, 27.5, 28.5, and 32 degrees.

2. The polymorph of claim 1 wherein the polymorph has at least six XRPD spectrum peaks selected from the group consisting of Two-Theta angles of approximately 7.5, 9, 11, 15.5, 27.5, 28.5, and 32 degrees.

3. The polymorph of claim 2, wherein the polymorph has XRPD spectrum peaks at Two-Theta angles of approximately 7.5, 9, 11, 15.5, 27.5, 28.5, and 32 degrees.

4. A pharmaceutical composition comprising a therapeutically effective amount of a polymorph of claim 1, and a pharmaceutically acceptable excipient or salt.

5. A method for the treatment or prophylaxis of a condition in a mammal in which copper delivery prevents, alleviates or ameliorates the condition comprising administering to the mammal a therapeutically effective amount of the composition of claim 4.

6. The method of claim 5, wherein the condition is selected from the group consisting of adriamycin-induced cardiomyopathy; AIDS dementia and HV-1 induced neurotoxicity; Alzheimer's disease; acute intermittent porphyria; Alzheimer's disease (AD); amyotrophic lateral sclerosis (ALS); atherosclerosis; cataract; cerebral ischemia; cerebral palsy; cerebral tumor; chemotherapy-induced organ damage; cisplatin-induced nephrotoxicity; coronary artery bypass surgery; Creutzfeldt-Jacob disease and its new variant associated with “mad cow” disease; diabetic neuropathy; Down syndrome; drowning; epilepsy and post-traumatic epilepsy; Friedrich's ataxia; frontotemporal dementia; glaucoma; glomerulopathy; hemochromatosis; hemodialysis; hemolysis; hemolytic uremic syndrome (Weil's disease); Lewy body dementia, Menkes disease; hemorrhagic stroke; Hallerboden-Spatz disease; heart attack and reperfusion injury; Huntington's disease; Lewy body disease; intermittent claudication; ischemic stroke; inflammatory bowel disease; macular degeneration; malaria; methanol-induced toxicity; meningitis (aseptic and tuberculous); motor neuron disease; multiple sclerosis; multiple system atrophy; myocardial ischemia; neoplasia; Parkinson's disease; peri-natal asphyxia; Pick's disease; progressive supranuclear palsy (PSP); radiotherapy-induced organ damage; restenosis after angioplasty; retinopathy; senile dementia; schizophrenia; sepsis; SCN2A-related epileptic encephalopathy; septic shock; spongiform encephalopathies; subarachnoid hemorrhage/cerebral vasospasm; subdural hematoma; surgical trauma, including neurosurgery; thalassemia; transient ischemic attack (TIA); synucleinopathies; transplantation; vascular dementia; viral meningitis; viral encephalitis; Neuropathies, acrodermatitis enteropathica; dementia with lewy bodies; tauopathies; mild cognitive impairment (MCI); motor neuron disease (MND) and prion disease.

7. The method of claim 6, wherein the condition is a neurodegenerative disease selected from the group consisting of Alzheimer's disease (AD), amyotrophic lateral sclerosis (ALS), Menkes disease, multiple sclerosis, neuropathies, motor neuron disease (MND), Parkinson's disease, Huntington disease, frontotemporal dementia, acrodermatitis enteropathica, Lewy body dementia, synucleinopathies, tauopathies, mild cognitive impairment (MCI), progressive supranuclear palsy (PSP) and prion disease.

8. A process for the preparation of a stable polymorph of CuATSM and gluconic acid, the process comprising the steps of:
a. mixing ATSMH₂ and copper gluconate (in a first ratio) with a solvent (in a second ratio) to form a slurry;
b. heating the slurry to form a composition comprising CuATSM and gluconic acid; and
c. isolating the polymorph.

9. The process of claim 8 wherein the solvent is selected from the group consisting of heptane, isopropyl acetate, and a heptane/isopropyl acetate mixture.

10. The process of claim 9 wherein the solvent is heptane.

11. The process of claim 9 wherein the solvent is a heptane/isopropyl acetate mixture.

12. The polymorph of claim 1 made by a method comprising the steps of:
a. mixing ATSMH₂ and copper gluconate (in a first ratio) with a solvent (in a second ratio) to form a slurry;
b. heating the slurry to form the composition; and
c. isolating the polymorph.

13. The polymorph of claim 12 wherein the solvent is selected from the group consisting of heptane, isopropyl acetate, and a heptane/isopropyl acetate mixture.

14. The polymorph of claim 13 wherein the solvent is heptane.

15. The polymorph of claim 13 wherein the solvent is a heptane/isopropyl acetate mixture.
